(19) United States
(12) Patent Application Publication
(10) Pub. No.: US 2005/0238727 A1
(43) Pub. Date: Oct. 27, 2005

(54) Coadministration of radiation, efaproxiral sodium, and supplemental oxygen for the treatment of cancer

(76) Inventors: Pablo J. Cagnoni, Denver, CO (US); Adam P. Boyd, Louisville, CO (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 11/112,660

(22) Filed: Apr. 22, 2005

Related U.S. Application Data
(60) Provisional application No. 60/564,383, filed on Apr. 22, 2004.

(51) Int. Cl. A61K 33/00; A61N 5/00; A61K 31/195

(52) U.S. Cl. 424/600; 600/1; 514/563

(57) ABSTRACT

Disclosed is a method of treating a cancer of the central nervous system in a host including administering radiation to the host; and administering efaproxiral sodium plus supplemental oxygen to the host; wherein the radiation and efaproxiral sodium and supplemental oxygen are administered in amounts effective to cause the arrest or regression of the cancer of the central nervous system in the host.

### CROSS REFERENCE TO RELATED APPLICATIONS

This application claims the benefit, under 35 U.S.C. § 119, of U.S. Provisional Application Ser. No. 60/564,383, entitled “Coadministration of Radiation and RSR13 For the Treatment of Cancer,” filed Apr. 22, 2004, and incorporated by reference herein in its entirety.

### FIELD OF THE INVENTION

The invention relates to treatment of cancer, more particularly to coadministration of efaproxiral sodium supplemental oxygen, and radiation for treatment of cancer.

### BACKGROUND OF THE INVENTION

The brain, cranial nerves, leptomeninges, spinal cord, and eye compose the central nervous system (CNS) and are at risk for the development of metastases from cancers. Chang & Lo, Diagnosis and Management of Central Nervous System Metastases from Breast Cancer, (2003) The Oncologist, 8:398-410. The disclosure of Chang & Lo, and all other patents, patent applications, and publications referred to herein are incorporated by reference herein in their entirety. Multiple, large autopsy series suggest that, in order of decreasing frequency, lung, breast, melanoma, renal, and colon cancers are the most common primary tumors to metastasize to the brain. Conventional treatment is aimed at palliation of symptoms and preservation of neurologic function. Conventional whole brain radiation therapy has been the mainstay of palliative treatment for brain, cranial nerve, spinal cord, and ocular metastases.

Hypoxic tumors are more resistant to cell damage by radiation, and tumor hypoxia adversely affects the clinical prognosis of RT.12-16 Oxygen measurements in human tumors have confirmed tumor hypoxia in brain metastases, glioblastoma multiforme (GBM), squamous cell carcinomas of the uterine cervix, head and neck, and breast carcinoma. Hypoxic tumors are substantially more resistant to radiation than oxygenated tumors, even small hypoxic fractions in a tumor may affect the overall response to RT, and increase the probability that some tumor cells will survive. Conversely, few hypoxic cells exist in normal tissue. Therefore, the toxicity of RT to normal tissue is not expected to be increased by therapies that increase 02 delivery to this small fraction of hypoxic cells.

Other treatment options for brain metastases include surgery to resect brain metastases, and stereotactic radiosurgery (SRS) to focally irradiate metastases. Ongoing research is aimed at refining criteria to select which patients with brain metastases should undergo surgery and SRS and how these focal therapies should be optimally integrated with whole-brain radiotherapy. Despite advances in neuroimaging, surgery, and radiation therapy, novel treatments are needed to improve the effectiveness of treatments for CNS metastases.

### SUMMARY OF THE INVENTION

The present invention provides methods of treating a central nervous system metastatic cancer sensitive to the combination of radiation, supplemental oxygen, and efaproxiral sodium in a host having a central nervous system metastatic cancer. In one embodiment, the method comprises, administering radiation to the host; administering efaproxiral sodium to the host; and administering supplemental oxygen to the host, wherein the radiation, supplemental oxygen, and efaproxiral sodium are administered in amounts effective to cause an arrest or regression of the central nervous system cancer in the host.

In one embodiment, the administration of efaproxiral sodium is at a dosage selected from the group consisting of
i) 100 mg/kg, if conditions are conditions selected from the group consisting of:
   a) radiation treatment day 1, the host is a male≦95 kg, and SpO2 is≧93%
   b) radiation treatment day 1, the host is a female≦70 kg, and SpO2 is≧93%
   c) radiation treatment day 2-10, the dose was 75 mg/kg on the previous dosing day, and SpO2 while breathing room air is currently≧93% and no adverse event occurred on the previous dosing day, wherein said adverse event is selected from the group consisting of supplemental oxygen administration>3 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, and the patient developed hypoxemia which required treatment after discharge on the previous dosing day;
   d) radiation treatment day 2-10, SpO2 is>90%, the dose was 100 mg/kg on the previous day and no adverse event occurred on the previous day, wherein said adverse event is selected from the group consisting of supplemental oxygen administration>3 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, the patient developed hypoxemia which required treatment after discharge on the previous dosing day, and SpO2 while breathing room air is 90-92% but was≧93% on the previous dosing day;
ii) 75 mg/kg, if conditions are conditions selected from the group consisting of:
   a) radiation treatment day 1, the host is a male>95 kg, and SpO2 is≧93%,
   b) radiation treatment day 1, the host is a female>70 kg, and SpO2 is≧93%,
   c) radiation treatment day 1 and SpO2 is 90-92%,
   d) radiation treatment day 2-10, the previous day's dose was held, SpO2 is 90-92% and SpO2 was 90-92% on the dosing day that led to holding the efaproxiral sodium dose,
   e) radiation treatment day 2-10, the previous day's dose was held, and SpO2 is≧93%,
   f) radiation treatment day 2-10, the previous day's dose was 100 mg/kg, and an adverse event occurs, wherein said adverse event is selected from the group consisting of supplemental oxygen administration>3 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, the patient developed hypoxemia which required treatment after discharge on the previous dosing day, and SpO2 while breathing room air is 90-92% but was≧93% on the previous dosing day, and
   g) radiation treatment day 2-10, SpO2 is>90%, and the dose was 75 mg/kg on the previous day and no adverse event occurred on the previous day, wherein said adverse event is selected from the group consisting of supplemental oxygen administration>3 hours after end-infusion of efaproxiral sodium before SpO2 while breathing room air returned to ≧90% on the previous dosing day, the patient experienced nausea and/or vomiting (grade 2 or higher) or clinically significant hypotension associated with efaproxiral sodium within 12 hours after efaproxiral sodium administration on the previous dosing day, the patient developed hypoxemia which required treatment after discharge on the previous dosing day, and SpO2 while breathing room air is 90-92% but was≧93% on the previous dosing day; and

iii) 0 mg/kg, if conditions are conditions selected from the group consisting of:
a) SpO2 is<90%,
b) radiation treatment day 2-10, the dose was 75 mg/kg on the previous day and an adverse event occurs, wherein said adverse event is selected from the group consisting of supplemental oxygen administration>3 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, the patient developed hypoxemia which required treatment after discharge on the previous dosing day, and SpO2 while breathing room air is 90-92% but was≧93% on the previous dosing day,
c) radiation treatment day 2-10, the dose was 0 mg/kg on the previous day, SpO2 is 90-92% but had been≧93% on the previous dosing day that led to holding efaproxiral sodium
d) radiation treatment day 2-10, SpO2 is>90%, and the dose was 0 mg/kg on the previous day and an adverse event occurs, wherein said adverse event is selected from the group consisting of supplemental oxygen administration>3 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, the patient developed hypoxemia which required treatment after discharge on the previous dosing day, and SpO2 while breathing room air is 90-92% but was≧93% on the previous dosing day.

In another embodiment, the host has breast cancer and a central nervous system metastatic cancer and the administration of efaproxiral sodium is at a dosage selected from the group consisting of
i) 75 mg/kg, if conditions are conditions selected from the group consisting of:
   a) radiation treatment day 1, SpO2 is≧90%, and creatinine≦2.0 mg/dL;
   b) radiation treatment day 4-10, the previous day's dose was 100 mg/kg, and an adverse event occurred on the previous day, wherein said adverse event is selected from the group consisting of supplemental oxygen administration≧4 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, and the patient SpO2 while breathing room air is 90-92% and has decreased from a baseline of≧93% on the previous dosing day; and
   c) radiation treatment day 2-10, SpO2 is≧90%, and the dose was 75 mg/kg on the previous day and no adverse event occurred on the previous day, wherein said adverse event is selected from the group consisting of ssupplemental oxygen administration≧4 hours after end-infusion of efaproxiral sodium before SpO2 while breathing room air returned to ≧90% on the previous dosing day, the patient experienced nausea and/or vomiting (grade 2 or higher) or clinically significant hypotension associated with efaproxiral sodium within 12 hours after efaproxiral sodium administration on the previous dosing day, and the patient SpO2 while breathing room air is 90-92% and has decreased from a baseline of≧93% on the previous dosing day; and
ii) 100 mg/kg, if conditions are radiation treatment day 3-10, the dose was 75 mg/kg on the previous two dosing days or 100 mg/kg on the previous dosing day, and SpO2 while breathing room air is≧90% and no adverse event occurred on the previous dosing day, wherein said adverse event is selected from the group consisting of supplemental oxygen administration≧4 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, and the patient SpO2 while breathing room air is 90-92% and has decreased from a baseline of≧93% on the previous dosing day; and
iii) 0 mg/kg, if conditions are conditions selected from the group consisting of:
   a) SpO2 is<90%,
   b) creatinine is>2.0 mg/dL;
   c) the patient developed hypoxemia which required treatment on the previous treatment day;
   d) RT day 2-10, the dose was 75 mg/kg on the previous day and an adverse event occurred, wherein said adverse event is selected from the group consisting of supplemental oxygen administration>3 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, the patient developed hypoxemia which required treatment after discharge on the previous dosing day, and SpO2 while breathing room air is 90-92% but was≧93% on the previous dosing day.

In some embodiments, the radiation is administered in at least about 3 Gray (Gy) fractions at least once every day for ten days to a treatment volume. In some embodiments, the radiation is administered in fractions, wherein 10 fractions are administered to an initial treatment volume. In some embodiments, a total of at least about 30 Gy of radiation is administered to the host. In some embodiments, radiation is administered to a whole brain of the host.

In some embodiments, the efaproxiral sodium is administered via a route selected from the group consisting of intravenously, including via a central venous access device, or via a peripheral route, via continuous infusion, and intraarterially. In some embodiments, the efaproxiral sodium is administered at an initial dosing level of at least about 75 mg/Kg/day. In some embodiments, the efaproxiral sodium is administered so as to achieve a RBC concentration of greater than about 483 μg/ml. In some embodiments, the metastatic cancer is derived from a primary cancer selected from the group consisting of lung, breast, melanoma, renal, and colon.

### BRIEF DESCRIPTION OF THE FIGURES

FIG. 1 shows the dosing algorithm for efaproxiral sodium on Day 1 of radiation treatment.

FIG. 2 shows the dosing algorithm for efaproxiral sodium on Day 2 of radiation treatment.

FIG. 3 shows the dosing algorithm for efaproxiral sodium on Days 3-10 of radiation treatment.

FIG. 4 shows the dosing algorithm for efaproxiral sodium on Days 1-2 of radiation treatment for patients with brain metastases from breast cancer.

FIG. 5 shows the dosing algorithm for efaproxiral sodium on Days 3-10 of radiation treatment for patients with brain metastases from breast cancer.

### DETAILED DESCRIPTION OF THE INVENTION

It has been discovered that efaproxiral sodium (sometimes referred to as RSR13) may be administered, together with radiation and supplemental oxygen, in the treatment of cancers of the central nervous system, wherein the supplemental oxygen, radiation and efaproxiral sodium are administered in amounts effective to treat the cancer of the central nervous system in the host. Generally, an effective amount is an amount effective to either (1) reduce the symptoms of the disease sought to be treated or (2) induce a pharmacological change relevant to treating the disease sought to be treated. For cancer, an effective amount includes an amount effective to: reduce the size of a tumor; slow the growth of a tumor; prevent or inhibit metastases; increase the life expectancy of the affected individual; or stabilize or improve the quality of life of the affected individual. In some embodiments, the cancer of the central nervous system is a metastatic cancer. In some embodiments, the primary cancer that has metastasized is a lung, breast, melanoma, renal, or colorectal cancer.

Efaproxiral sodium is 2-[4-(((3,5-dimethylanilino)carbonyl)methyl)phenoxy]-2-methylpropionic acid: 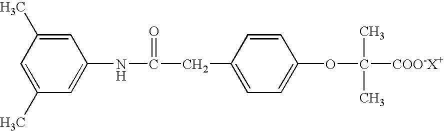
is an allosteric effector of hemoglobin, and has been shown to enhance tissue oxygenation in vivo. Sometimes, efaproxiral sodium is represented by the name 2-[4-[2-[(3,5-dimethylphenyl)amino]-2-oxoethyl]phenoxy]-2-methylpropanoic acid. In general efaproxiral sodium is administered as a physiologically acceptable salt, such as the monosodium salt; that is, X+ is Na+. Efaproxiral sodium induces allosteric modification of hemoglobin, such that its binding affinity for oxygen is decreased, resulting in increased oxygen distribution to tissues by erythrocytes.

The ability to allosterically modify hemoglobin also allows the compounds to be useful in treating a variety of disorders and conditions mediated through allosterically modifying hemoglobin to shift oxygen equilibrium in favor of free oxygen. Such disorders may include, but are not limited to, whole body or tissue hypothermia, hypoxia or hypotension, wounds, brain injury, diabetic ulcers, chronic leg ulcers, pressure sores, tissue transplants, stroke or cerebro ischemia, ischemia or oxygen deprivation, respiratory disorders including acute respiratory distress syndrome and chronic obstructive pulmonary disorder, surgical blood loss, sepsis, multi-system organ failure, normovolemic hemodilution procedures, carbon monoxide poisoning, bypass surgery, carcinogenic tumors, oxygen deprivation of a fetus. The compound is useful in a method comprising the step of administering to a patient suffering from or undergoing the claimed condition, a sufficient quantity of an allosteric effector compound. In the case of carcinogenic tumors, the compound is useful as a radiosensitizer in conjunction with ionizing radiation (See Teicher, (1996) Drug Dev. Res. 38:1-11; Rockwell and Kelley (1998) Rad. Oncol. Invest. 6:199-208; and Khandelwal et al. (1996) Rad. Oncol. Invest. 4:51-59). The allosteric modification properties also allow it to be useful in certain imaging methods, especially blood oxygen level dependent MRI, and also in diagnostic methods, including determination of tumor oxygenation, and determination of an optimal time for commencing radiation treatment based on tumor oxygenation. The preparation and uses for 2-[4-[2-[(3,5-dimethylphenyl)amino]-2-oxoethyl]phenoxy]-2-methyl-propionic acid and its physiologically acceptable salts has been described previously in U.S. Pat. Nos. 5,049,695; 5,122,539; 5,290,803; 5,432,191; 5,525,630; 5,648,375; 5,661,182; 5,677,330; 5,705,521; 5,872,888; and 5,927,283, and U.S. Patent Application Publication No. 20030017612 A1. These patents also describe the preparation and use of structurally similar compounds. Other patents describing closely related compounds include U.S. Pat. Nos. 5,248,785 and 5,731,454. These patents, applications, and all other patents, applications, and publications referred to herein, are specifically incorporated by reference herein.

In general, the invention provides a course of whole brain radiation therapy (WBRT) with supplemental oxygen and efaproxiral sodium. In one embodiment, the WBRT is a multi-day, fractionated course of WBRT. In one embodiment, the course is a 10-day course. In one embodiment, efaproxiral sodium and supplemental oxygen is received within about 30 minutes prior to daily WBRT. In this embodiment, efaproxiral sodium administration with supplemental oxygen begins on the first day of radiation treatment (RT day 1) and will continue every RT day during the 10-day course of WBRT, for a total of 10 doses.

In general, efaproxiral sodium is administered in an initial dose of about 75-100 mg/kg. In one embodiment, subsequent doses of efaproxiral sodium are 75-100 mg/kg. In another embodiment, subsequent doses of efaproxiral sodium are determined with reference to standard cutaneous pulse oximetry (SpO2) and the presence of pharmacologic effects on blood oxygen saturation. The efaproxiral sodium dose may be lowered to 0-75 mg/kg if unacceptable nausea, vomiting, hypoxemia, or low blood oxygen saturation (SpO2) events are observed. The efaproxiral sodium dose may be increased to 75-100 mg/kg if the SpO2 is normal, at baseline or improved, and no unacceptable nausea, vomiting, or hypoxemia events occurred on the previous day. In some embodiments, doses of efaproxiral sodium are determined with reference to creatinine levels. The efaproxiral sodium dose may be lowered to 0 mg/kg if creatinine is>2.0 mg/dL on any scheduled RT day. In general, efaproxiral sodium is administered by intravenous infusion through a central venous access device over 30-45 minutes.

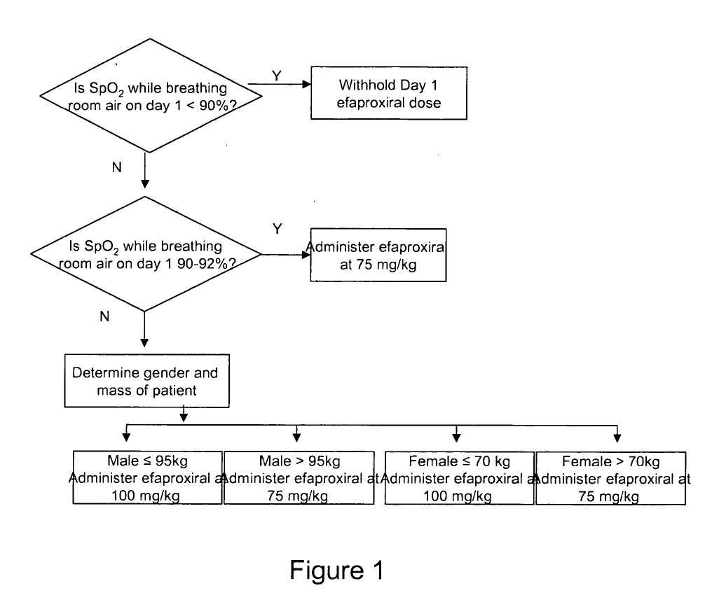

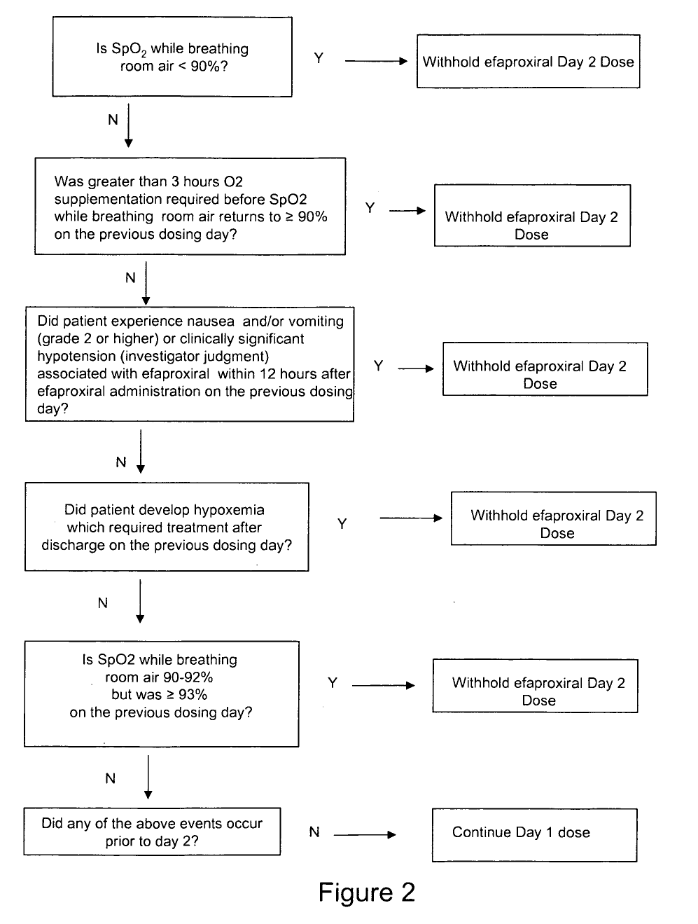

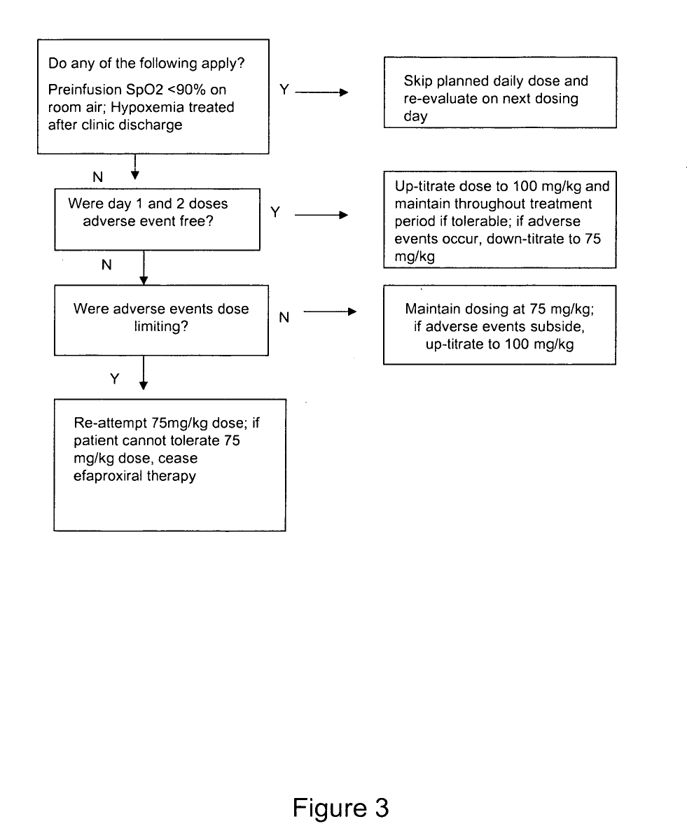

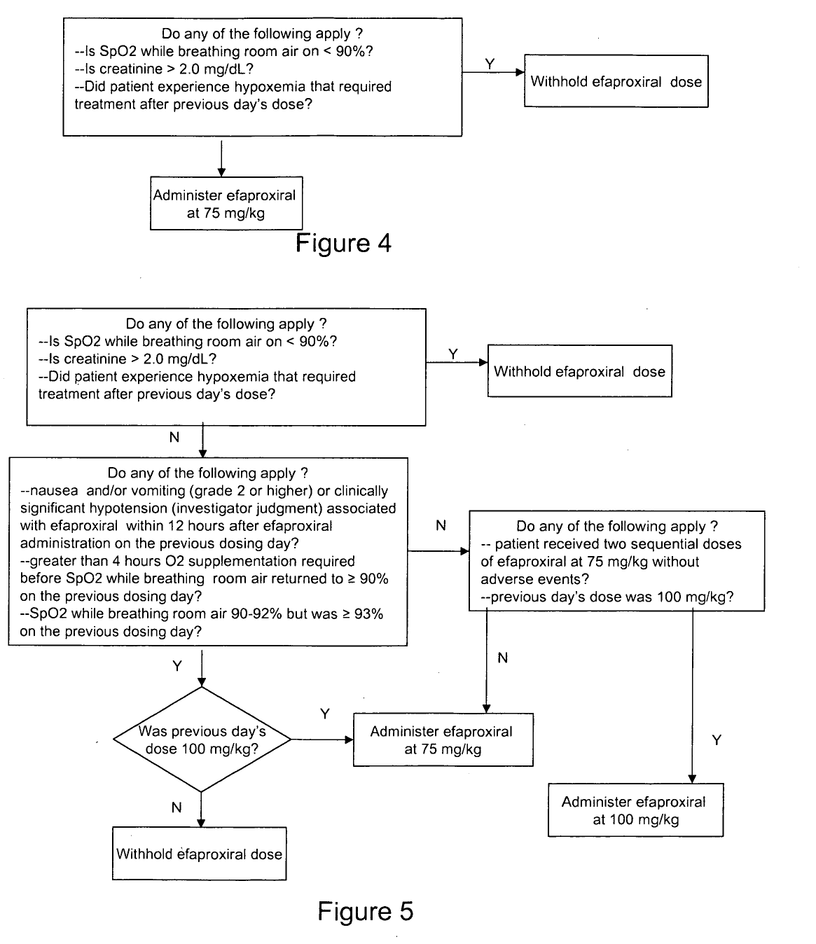

In one embodiment, the invention provides a 10-day course of WBRT with supplemental oxygen and efaproxiral sodium, wherein the efaproxiral sodium is administered as shown in FIG. 1 on RT day 1, and is administered as shown in FIG. 2 on RT day 2, and is administered as shown in FIG. 3 on days 3-10. In one embodiment, where the patient has breast cancer, the invention provides a 10-day course of WBRT with supplemental oxygen and efaproxiral sodium, wherein the efaproxiral sodium is administered as shown in FIG. 4 on RT day 1-2, and is administered as shown in FIG. 5 on RT day 3-10.

Patients treated with efaproxiral sodium received supplemental oxygen via a mask or nasal cannula. Efaproxiral sodium decreases hemoglobin oxygen binding affinity and reduces oxygen loading in the lungs at ambient oxygen pressure. Without being bound by theory, it is believed that the administration of supplemental oxygen serves to optimize both hemoglobin oxygen saturation and tumor oxygenation, and to assure pulmonary oxygen uptake. In one embodiment, supplemental oxygen is administered for at least about 30 minutes prior to, during, and for at least 15 minutes after completion of daily WBRT. In another embodiment, supplemental oxygen is administered for at least about 5 minutes prior to, during, and for at least 15 minutes after completion of daily WBRT. In one embodiment, the dose of supplemental oxygen is 4L/minute. In another embodiment, the dose of supplemental oxygen is adjusted as needed to maintain an SpO2 measurement of greater than or equal to 90% during and after efaproxiral sodium administration. The oxygen may be 100% oxygen, carbogen, or other suitable exogenous oxygen source.

Radiation may be administered in a variety of fashions. For example, radiation may be electromagnetic or particulate in nature. Electromagnetic radiation useful in the practice of this invention includes, but is not limited, to x-rays and gamma rays. Particulate radiation useful in the practice of this invention includes, but is not limited to, electron beams, proton beams, neutron beams, alpha particles, and negative pi mesons. The radiation may be delivered using conventional radiological treatment apparatuses and methods, and by intraoperative and stereotactic methods. Additional discussion regarding radiation treatments suitable for use in the practice of this invention may be found throughout Steven A. Leibel et al., Textbook of Radiation Oncology (1998) (publ. W. B. Saunders Company), and particularly in Chapters 13 and 14. Radiation may also be delivered by other methods such as targeted delivery, for example by radioactive “seeds,” or by systemic delivery of targeted radioactive conjugates. J. Padawer et al., Combined Treatment with Radioestradiol lucanthone in Mouse C3HBA Mammary Adenocarcinoma and with Estradiol lucanthone in an Estrogen Bioassay, Int. J. Radiat. Oncol. Biol. Phys. 7:347-357 (1981). Other radiation delivery methods may be used in the practice of this invention. [0024] The amount of radiation delivered to the desired treatment volume may vary. In one embodiment, radiation may be administered in amounts effective to cause the arrest or regression of the cancer of a central nervous system in a host, when the radiation is administered with efaproxiral sodium and supplemental oxygen. In one embodiment, radiation is administered in at least about 3 Gray (Gy) fractions at least once per day for five days per week, over ten days, to a treatment volume of up to about 30 Gray (GY). In other embodiments, different hyper-fractionated radiation schemes known to those skilled in the art are deployed such as 15 administrations of 2 Gy fractions or 12 administrations of 2.5 Gy fractions.

When irradiating the whole brain, a maximum dosage of 30 Gy is recommended. In one embodiment, radiation is administered to the whole brain of a host, wherein the host is being treated for metastatic cancer. In one embodiment, radiation is administered as soon as possible, or about within 30 minutes maximum, post-end efaproxiral sodium administration.

It will be apparent to those skilled in the art that various modifications and variations can be made in the methods of the present invention without departing from the spirit or scope of the invention. Thus, it is intended that the present invention cover the modifications and variations of this invention provided they come within the scope of the appended claims and their equivalents. Additionally, the following examples are appended for the purpose of illustrating the claimed invention, and should not be construed so as to limit the scope of the claimed invention.

### EXAMPLES

### Example 1

### Materials and Methods

A. Efaproxiral Forumulation

Efaproxiral sodium has been formulated as a sterile solution for injection and will be supplied in single-use 500 mL glass bottles containing 10 grams of efaproxiral in 500 mL of 0.225% sodium chloride (NaCl) and 1.0 mM phosphate buffer, pH 7.5. The osmolality of efaproxiral injection ( efaproxiral) is approximately equivalent to 0.45% NaCl (half-normal saline). The stock efaproxiral solution is infused directly from the bottle at the original concentration of 20 mg/mL (no dilution). The dosing weight for patients will be a weight obtained at the baseline visit. The volume to be infused will be based on the following calculation

$$\frac{( Patient weight [ kg ] ) \times ( efaproxiral dose [ mg \text{/} kg ] )}{20 mg \text{/} mL ( efaproxiral concentration )}$$

Before administration, efaproxiral bottles will be inspected for abnormalities such as cracks, sediments, crystals, turbidity, etc. Efaproxiral will be administered IV via a CVAD.

B. Administration Methods

Record resting SpO2 prior to administering supplemental 02. Administer 4 L/minute supplemental 02 by nasal cannula (NC).

A CVAD, preferably a peripherally inserted central catheter (PICC), is useful for administration of efaproxiral. If a patient has a pre-existing CVAD, it must be assessed for patency and adequacy of usage for efaproxiral administration. Patients will be assessed frequently for any adverse sequelae due to CVADs such as thrombosis or infection associated with chronic catheterization. Efaproxiral administration will begin on WBRT day 1. Efaproxiral will be infused through the CVAD over 30 minutes at a constant rate via a volumetric pump. If the infusion of efaproxiral is interrupted or prolonged, then the infusion will be continued but should not exceed 45 minutes. WBRT should start as soon as possible after completion of the efaproxiral infusion, but must begin within 30 minutes. If the SpO2 while breathing room air prior to receiving supplemental 02 and efaproxiral is<90%, or creatinine>2.0 mg/dL on any WBRT day, omit efaproxiral for the scheduled treatment day. Patients will receive supplemental oxygen starting 5 minutes prior to efaproxiral, during efaproxiral, during WBRT, and for at least 15 minutes after the end of WBRT. If efaproxiral is omitted on any WBRT day based on clinical or SpO2 criteria, the patient should receive WBRT alone with supplemental 02 for at least 35 minutes prior to, during, and at least 15 minutes after WBRT. Efaproxiral doses that are omitted will not be made up.

The procedures listed below will be performed every WBRT day. Ensure patient has an appropriate resting SpO2 prior to starting supplemental 02. Start supplemental 02 at least 5 minutes prior to starting the efaproxiral infusion. Administer efaproxiral over 30 minutes, using a volumetric pump, via the CVAD. Monitor the patient by clinical observations. Administer WBRT within 30 minutes after the end of efaproxiral infusion. All SpO2 measurements while breathing room air should be obtained after supplemental 02 has been discontinued for at least 5 minutes. The first SpO2 measurement obtained while breathing room air (for at least 5 minutes) should be obtained within 20 minutes after the end of WBRT. If it is not possible to obtain this measurement in the time frame indicated, obtain it as soon as possible after the completion of WBRT. If the SpO2 while breathing room air is below 90%, obtain the SpO2 measurement, and immediately re-administer supplemental O2 for an additional 30 minutes. Discontinue supplemental O2 for at least 5 minutes and obtain another SpO2 measurement while breathing room air.

If the SpO2 while breathing room air is maintained at≧90% during the initial 5-minute period, proceed.

Monitor and assure that the SpO2 while breathing room air is maintained at≧90% for at least an additional 15 minutes by taking a second reading, while breathing room air, at the end of the 15-minute period. If the SpO2 while breathing room air was maintained at≧90% for at least 15 minutes.

### Example 2

### Dosing Guidelines for a Weight- and Gender-Based Method

Dosing of efaproxiral sodium is determined as follows. Table 1 illustrates the efaproxiral sodium initial dose schedule.

TABLE 1
Initial Dose Calculator

| Gender | Body weight category | SpO₂ ≧ 93% | SpO₂ 90-92% |
|---|---|---|---|
| Female | ≦70 kg | 100 mg/kg | 75 mg/kg |
| | >70 kg | 75 mg/kg | 75 mg/kg |
| Male | ≦95 kg | 100 mg/kg | 75 mg/kg |
| | >95 kg | 75 mg/kg | 75 mg/kg |

Depending upon an individual patient's resaturation time (time required to recover to a stable≧90% SpO2 on room air) following efaproxiral sodium plus WBRT, supplemental oxygen use may be required for as little as 30 minutes to more than 4 hours. The majority of efaproxiral sodium doses in patients with brain metastases from breast cancer required one hour or less of supplemental oxygen after the completion or WBRT. During this period of decreased oxygen saturation, patients require continuous SpO2 monitoring. If the desired SpO2 of≧90% while breathing room air is not achieved, supplemental oxygen is to be continued and increased to a flow of 6-8 L/min, if necessary, until the SpO2 while breathing room air is stabilized at≧90%.

Dose Modifications—Dosage adjustment is based upon clinical assessments and monitoring of SpO2 indicating that the patient is experiencing exaggerated effects or toxicities. Table 2 summarizes the efaproxiral sodium weight and gender-based dose modification schedule.

TABLE 2
Calculator for Subsequent Efaproxiral Sodium Doses

| Evaluations Prior to Each Treatment Day | Efaproxiral Sodium Dose |
|---|---|
| SpO₂ during infusion < 90% | DL − 1 |
| Pretreatment SpO₂ < 90% | Omit dose for current |
| | treatment day; when |
| | SpO₂ ≧ 90%, resume |
| | treatment at DL − 1 |
| Hypoxemia temporally | DL − 1 |
| associated with other | |
| signs/symptoms^{a} | |
| Renal dysfunction > Grade 1 | DL − 1 |
| Common Toxicity Criteria | |
| (CTC)^{b} | |
| Renal dysfunction > Grade 2 CTC^{c} | Permanently discontinue |
| | efaproxiral sodium |
| Pretreatment SpO₂ ≧ 93% | DL + 1 |
| on room air and ≧90% during | |
| efaproxiral sodium infusion on | |
| previous day without hypoxemia | |

^{a}Dyspnea, hypotension/dizziness, renal dysfunction (≧Grade 2 CTC or increase of 1 CTC Grade from baseline);
^{b}>Grade 1 CTC or 1 CTC Grade increase from baseline; CTC is based on National Cancer Institute (NCI) Toxicity Criteria scale Version 2.0 published 30 Apr 1999.
^{c}>Grade 2 CTC or increase of >1 CTC Grade from baseline.
DL + 1 Dose increase from 75 mg/kg to 100 mg/kg (max. dose) no further escalation beyond 100 mg/kg
DL − 1 Dose reduction from 100 mg/kg to 75 mg/kg, if current dose level is 75 mg/kg no further reduction beyond 75 mg/kg, instead omission of dose and resume treatment at 75 mg/kg on Treatment day (RT-day) + 1.
Efaproxiral sodium is administered via parenteral routes of administration, including but not limited to, intravenously, including via a central venous access device, or via a peripheral route, via continuous infusion, and intraarterially.

### Example 3

### Treatment Protocol

Patients with brain metastases were administered efaproxiral sodium in a total dose of 0-100 mg/kg per day based on the dosing guidelines detailed above. In general, efaproxiral sodium is administered by intravenous infusion through a central venous access device over 30 minutes at a dose of 75 or 100 mg/kg daily with concurrent supplemental oxygen. Oxygen must be administered at 4 L/min via nasal cannula or facemask beginning 5 minutes prior to initiation of infusion, during infusion and WBRT, and for at least 15 minutes after completion of daily WBRT. Efaproxiral sodium is administered every day of a fractionated course of WBRT. Except when contraindicated, WBRT must be administered within 30 minutes of the end of the efaproxiral sodium infusion.

The patients were given the drug in one dose. Efaproxiral sodium was administered via central venous access with flow rate controlled by volumetric pump over a 30-45 minute interval with end-infusion no longer than 30 minutes prior to each radiation dose of a 10-day course of WBRT. Efaproxiral sodium was formulated as a sterile solution for injection and was supplied in single-use glass bottles containing 10 g efaproxiral sodium in 500 mL of 0.225% NaCl at a concentration of 20 mg/mL. Efaproxiral sodium was administered during the 10-day course of WBRT, for a maximum of 10 doses. A control group received radiation and supplemental oxygen only.

Supplemental oxygen is administered at about 4 L/min via nasal cannula beginning about 5 minutes prior to initiation of infusion, during infusion and WBRT, and for at least about 15 minutes after completion of daily WBRT. If the desired SpO2 of greater than or equal to 90% while breathing room air is not achieved, supplemental oxygen is to be continued and increased to a flow of 6-8 L/min, if necessary, until the SpO2 while breathing room air is stabilized at greater than or equal to 90%.

Data obtained in the controlled study confirmed the previously suggested safety profile of efaproxiral sodium as sole adjunct to radiation therapy in the treatment of cancer patients. The majority of treatment-emergent adverse events were Grade 1 or 2 in severity, resolved spontaneously or within the duration of the study, and responded well to concomitant treatment with antiemetics for nausea/vomiting, nonsteroidal anti-inflammatory drugs for headache, supplemental oxygen for hypoxemia. While the most commonly reported Grade 3 adverse event in efaproxiral sodium-treated patients was hypoxemia (reported in 11 % of patients), no Grade 4 hypoxemia was reported. Muscle weakness and dyspnea (reported in 3% of patients ) were the most commonly reported Grade 3 adverse events in Control patients and both events were reported as a Grade 4 event in 1 patient each. The most commonly reported Grade 4 adverse event in both treatment and control groups was disease progression (reported in 6% of both groups).

### Example 4

### Pharmacokinetics

Plasma and red blood cell (RBC) drug concentration were assayed on 2 days during the course of efaproxiral sodium administration: WBRT day 1 (end-infusion) and on 1 single day between WBRT days 6 and 10 (pre-infusion and end-infusion assays). Regression analysis was used to explore the relationship between trough and peak concentrations and continuous clinical covariates (eg, age, serum albumin, hematocrit, serum creatinine, etc). No clinically relevant drug accumulation occurred based on WBRT week 2 preinfusion efaproxiral sodium concentrations in RBCs. A dose of efaproxiral sodium was considered predicatably therapeutic if it achieved a sufficient, RBC concentration (>483 μg/ml), and corresponded to a predicted p50 shift of 10 mmHg.

There was a proportional increase in the efaproxiral sodium concentrations in RBCs for patients dosed at 75 or 100 mg/kg. Patients with higher body weight had higher efaproxiral sodium concentrations in RBCs than low weight patients at a given dose. For all efaproxiral sodium-treated patients, those with a dose change had a higher efaproxiral sodium concentration in RBCs at the initial dose of 100 mg/kg than patients who had a starting dose of 100 mg/kg with no dose change. Efaproxiral sodium concentrations in RBCs were higher in breast primary patients than patients with NSCLC and other primary because there were a higher proportion of high body weight breast primary patients. Efaproxiral sodium concentrations in RBCs were comparable for NSCLC patients at 100 mg/kg and breast patients at 75 mg/kg, but the efaproxiral sodium concentrations in RBCs for NSCLC patients at 75 mg/kg were substantially lower in breast patients at 75 mg/kg. These analyses reveal that patients with efaproxiral sodium concentrations in RBCs that reached optimal levels had a better outcome than those patients who did not. A clear correlation between RBC concentration, number of successful efaproxiral sodium+WBRT+supplemental oxygen doses, and MST was established.

| | Control | | efaproxiral sodium | | |
|---|---|---|---|---|---|
| | <7 | | | ≧7 efaproxiral | ≧7 efaproxiral |
| | WBRT | ≧7 WBRT | <7 efaproxiral | sodium Doses | sodium Doses/ |
| | Doses | Doses | sodium Doses | <7 Successful^{(a)} | ≧7 Successful^{(a)} |
| Patients | MST (n) | MST (n) | MST (n) | MST (n) | MST (n) p value (b) |
| All | 0.71 (10) | 4.47 (257) | 2.96 (53) | 4.93 (118) | 6.90 (100) 0.001 |
| NSCLC | 0.66 (4) | 4.47 (147) | 2.71 (30) | 4.73 (65) | 6.83 (53) 0.0011 |
| Breast | Unk. (2) | 4.57 (53) | 3.52 (13) | 7.33 (22) | 25.72 (25) 0.0002 |

^{(a)}a dose of efaproxiral sodium was considered successful if it achieved a sufficient RBC conentration (>483 μg/ml); this corresponds to a predicted p50 shift of 10 mmHg
(b): vs Control³ 7 WBRT doses
MST: median survival time

### Example 5

### Efficacy

A. Patient Survival

One measurement of efficacy is the survival in the total patient population. For eligible patients, the observed mean survival time for the control group was 4.37 months as compared to 5.39 months for the efaproxiral sodium treated group.

In patients with breast as the site of primary, there was a highly statistically significant difference detected for the survival distribution function in the treatment versus the control group (HR=0.552, p=0.0061). Analyses showed consistent results for breast cancer patients across all pre-specified covariates.

The estimated increase in radiographic response rate in all patients randomized to the efaproxiral sodium group was 7.9% (95% CI: −0.4%-16.3%) compared to the Control group (p=0.0609). In breast primary patients, logistic multiple regression showed efaproxiral sodium treatment effect to be statistically significant for predicting response (p=0.0209). The increase in response rate translated into prolonged survival.

B. Radiographic Tumor Progression

Radiographic progression is defined by radiographic criteria only, based on a blinded central review. Determination of radiographic tumor progression in the brain was based on contrast enhanced MRI or CT scans taken at screening and compared to follow-up scans taken 1 month after the end of WBRT, 3 months after the end of WBRT, and every 3 months thereafter until death. Maximum bi-dimensional measurements (x=transverse, y=antero-posterior) were used to compute the bi-dimensional product and for determination of response and radiographic progression. Time to radiographic tumor progression in the brain was reported by means of Kaplan-Meier estimates. Gray's test (Gray R. A class of K-sample tests for comparing the cumulative incidence of a competing risk. Annals of Statistics 1998;16:1141-1154) was used to compare cumulative incidence between treatment and control groups. Potential competing risks for radiographic progression in the brain included death without progression and loss to follow-up. The date of tumor progression is defined as the date of radiographic documentation that any treated lesion in the brain is enlarged by more than 25% in the bi-dimensional product. The reference to “any treated lesion” mans that the lesion was present prior to RT.

C. Quality of Life

Quality of life was determined by means of the Spitzer Questionnaire (SQ) and Kamofsky Performance Status (KPS) assessment. The tests were performed at baseline, at WBRT day 10, and at all routine follow-up visits. A sustained drop in the KPS score to less than 60 was defined as a significant drop. The 5 questions comprising the Spitzer Questionnaire were weighted evenly. For each evaluation with at least 3 out of 5 questions answered, an average score was computed for each patient. Questionnaires with fewer than 3 questions answered were treated as missing data. The protocol specified a sustained drop in the Spitzer Questionnaire score of 2 points constituted a significant drop.

Comparisons of QOL measures between treatment and control groups focused on 1-month, 3-month, 6-month, and 1 -year follow-up time-points and did not include WBRT day 10.

There was a highly statistically significant percentage of patients with stable or improving KPS scores over time in the efaproxiral sodium group versus the Control group _{χ}2=9.0096, p=0.0027).

TABLE
Numbers and Percentages of All Randomized Patients with Stable or
Improving KPS Scores over Time in Study RT-009^{a}

| | Control (N = 267) | efaproxiral sodium (N = 271) |
|---|---|---|
| Time | n (%) | n (%) |
| 1 month | 96 (36) | 119 (44) |
| 3 months | 49 (18) | 64 (24) |
| 6 months | 39 (15) | 49 (18) |
| 12 months | 10 (4) | 19 (7) |

^{a}p = 0.0027, Cochran-Mantel-Haenszel test with time (1, 3, 6, and 12 months) as strata

There was a trend toward a higher percentage of patients with stable or improving SQ scores over time in the efaproxiral sodium group versus the Control group (_{χ}2=3.4675, p=0.0626)( ).

TABLE
Numbers and Percentages of All Randomized Patients with Stable or
Improving SQ Scores over Time in Study RT-009^{a}

| | Control (N = 267) | efaproxiral sodium (N = 271) |
|---|---|---|
| Time | n (%) | n (%) |
| 1 month | 98 (37) | 115 (42) |
| 3 months | 55 (21) | 62 (23) |
| 6 months | 39 (15) | 43 (16) |
| 12 months | 15 (6) | 24 (9) |

^{a}p = 0.0626, Cochran-Mantel-Haenszel test with time (1, 3, 6, and 12 months) as strata

For patients with breast primary, there was a statistically significant difference detected in the distribution of KPS score categories between treatment groups at 6 months and 1 year (p=0.0046 and p=0.0070, respectively).

### Example 6

### Treatment Protocol for Patients with Brain Metastases from Breast Camcer using Uniform Initial Dose

WBRT will consist of 10 fractions of 3.0 Gy each, given 5 days per week, for a total of 30.0 Gy. WBRT will be delivered with a megavoltage linear accelerator with photon energies between 4 and 8 megavolts (MV) (preferred). Cobalt 60 is also acceptable. Source skin distance (SSD) techniques or source axis distance (SAD) techniques should be at least 80 cm. Electron, particle, photon, or implant boost is not used. The patient will be treated in the supine or other appropriate position. A head-holding device may be used to ensure adequate immobilization during therapy and ensure reproducibility. The treatment volume should include the whole brain. There will be “flash” anteriorly, superiorly, and posteriorly. The inferior border of the WBRT field will be at the C1-C2 interspace. This can be lowered to the C2-C3 interspace for patients with cerebellar or lower brainstem (pons, medulla) metastases if clinically indicated. There will be at least 1 cm margin inferior to the floor of the posterior fossa. The lens should be shielded from the direct beam at all times. Any concurrent RT treatment field must not overlap with the WBRT treatment field. Two opposed coaxial equally weighted beams will be used. The target dose will be

specified on the central ray at the mid-separation of the beams. The total dose will be 30.0 Gy at 3.0 Gy fractions per day, 5 days per week, over 10 days. The field is 30.0 Gy delivered to standard whole brain field.

Dosing Adjustment Guideline If any of the following conditions are present on any WBRT day, omit efaproxiral for the scheduled treatment day:

Preinfusion SpO2 while breathing room air<90%.

Creatinine>2.0 mg/dL (177 μmol/L).

Hypoxemia that required treatment after clinic discharge.

Patients may experience 1 or more of the following adverse events after efaproxiral administration that may warrant efaproxiral dose adjustment:

Required duration of supplemental O2 administration was≧4 hours after end-infusion before SpO2 while breathing room air was maintained at≧90%.

Nausea and/or vomiting (CTCAE Grade 2 or higher) or clinically significant hypotension associated with efaproxiral within 12 hours after efaproxiral administration.

Preinfusion SpO2 while breathing room air is currently 90-92%, and has decreased from a previous baseline SpO2≧93%.

Efaproxiral on WBRT days 1 and 2: Administer 75 mg/kg of efaproxiral on WBRT day 1.

If the patient did not experience any of the adverse events listed above after efaproxiral on WBRT day 1, administer 75 mg/kg of efaproxiral on WBRT day 2.

If the patient experienced any of the adverse events listed above after receiving efaproxiral on WBRT days 1 or 2, omit efaproxiral for the scheduled treatment day.

The patient must tolerate 2 sequential days of 75 mg/kg of efaproxiral before escalating the efaproxiral dose to 100 mg/kg.

Efaproxiral on WBRT days 3-10:

Administer 100 mg/kg of efaproxiral if the patient did not experience any of the Adverse events listed above after 2 sequential doses of 75 mg/kg.

If the patient does not experience any of the adverse events listed above after receiving 100 mg/kg of efaproxiral, remain on this dose for the duration of the treatment.

If the patient experiences any of the adverse events listed above after receiving 100 mg/kg of efaproxiral, reduce dose to 75 mg/kg and remain on this dose for the duration of the treatment.

If the patient experiences any of the adverse events listed above after receiving 75 mg/kg of efaproxiral, then omit efaproxiral for the scheduled treatment day.

1. A method of treating a central nervous system metastatic cancer sensitive to the combination of radiation, supplemental oxygen, and efaproxiral sodium in a host having a central nervous system metastatic cancer comprising:
administering radiation to the host;
administering efaproxiral sodium to the host, and
administering supplemental oxygen to the host,
wherein the radiation, supplemental oxygen, and efaproxiral sodium are administered in amounts effective to cause an arrest or regression of the central nervous system cancer in the host.

2. The method of claim 1, comprising:
A) administering radiation to the host;
B) administering efaproxiral sodium to the host, wherein the efaproxiral sodium is administered at a dosage selected from the group consisting of
i) 100 mg/kg, if conditions are conditions selected from the group consisting of:
a) radiation treatment day 1, the host is a male≦95 kg, and SPO2 is≧93%
b) radiation treatment day 1, the host is a female≦70 kg, and SPO2 is≧93%
c) radiation treatment day 2-10, the dose was 75 mg/kg on the previous dosing day, and SPO2 while breathing room air is currently≧93% and no adverse event occurred on the previous dosing day, wherein said adverse event is selected from the group consisting of supplemental oxygen administration>3 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, and the patient developed hypoxemia which required treatment after discharge on the previous dosing day;
d) radiation treatment day 2-10, SpO2 is>90%, the dose was 100 mg/kg on the previous day and no adverse event occurred on the previous day, wherein said adverse event is selected from the group consisting of supplemental oxygen administration>3 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, the patient developed hypoxemia which required treatment after discharge on the previous dosing day, and SpO₂ while breathing room air is 90-92% but was≧93% on the previous dosing day;
ii) 75 mg/kg, if conditions are conditions selected from the group consisting of:
a) radiation treatment day 1, the host is a male>95 kg, and SpO2 is≧93%,
b) radiation treatment day 1, the host is a female>70 kg, and SpO2 is≧93%,
c) radiation treatment day 1 and SpO2 is 90-92%,
d) radiation treatment day 2-10, the previous day's dose was held, SpO2 is 90-92% and SpO2 was 90-92% on the dosing day that led to holding the efaproxiral sodium dose,
e) radiation treatment day 2-10, the previous day's dose was held, and SpO2 is≧93%,
f) radiation treatment day 2-10, the previous day's dose was 100 mg/kg, and an adverse event occurs, wherein said adverse event is selected from the group consisting of supplemental oxygen administration>3 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, the patient developed hypoxemia which required treatment after discharge on the previous dosing day, and SpO2 while breathing room air is 90-92% but was≧93% on the previous dosing day, and
g) radiation treatment day 2-10, SpO2 is>90%, and the dose was 75 mg/kg on the previous day and no adverse event occurred on the previous day, wherein said adverse event is selected from the group consisting of supplemental oxygen administration>3 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, the patient developed hypoxemia which required treatment after discharge on the previous dosing day, and SpO₂ while breathing room air is 90-92% but was≧93% on the previous dosing day; and
iii) 0 mg/kg, if conditions are conditions selected from the group consisting of:
a) SpO2 is<90%,
b) radiation treatment day 2-10, the dose was 75 mg/kg on the previous day and an adverse event occurs, wherein said adverse event is selected from the group consisting of supplemental oxygen administration>3 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, the patient developed hypoxemia which required treatment after discharge on the previous dosing day, and SpO2 while breathing room air is 90-92% but was≧93% on the previous dosing day,
c) radiation treatment day 2-10, the dose was 0 mg/kg on the previous day, SpO2 is 90-92% but had been≧93% on the previous dosing day that led to holding efaproxiral sodium
d) radiation treatment day 2-10, SpO2 is>90%, and the dose was 0 mg/kg on the previous day and an adverse event occurs, wherein said adverse event is selected from the group consisting of supplemental oxygen administration>3 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, the patient developed hypoxemia which required treatment after discharge on the previous dosing day, and SpO2 while breathing room air is 90-92% but was≧93% on the previous dosing day; and
C) administering supplemental oxygen to the host,
wherein the radiation, supplemental oxygen, and efaproxiral sodium are administered in amounts effective to cause an arrest or regression of the central nervous system cancer in the host.

3. The method of claim 1, comprising:
A) administering radiation to a host having breast cancer and a central nervous system metastatic cancer and;
B) administering efaproxiral sodium to the host, wherein the efaproxiral sodium is administered at a dosage selected from the group consisting of
i) 75 mg/kg, if conditions are conditions selected from the group consisting of:
a) radiation treatment day 1, SpO₂ is≧90%, and creatinine≦2.0 mg/dL;
b) radiation treatment day 4-10, the previous day's dose was 100 mg/kg, and an adverse event occurred on the previous day, wherein said adverse event is selected from the group consisting of supplemental oxygen administration≧4 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, and the patient SpO2 while breathing room air is 90-92% and has decreased from a baseline of≧93% on the previous dosing day; and
c) radiation treatment day 2-10, SpO2 is>90%, and the dose was 75 mg/kg on the previous day and no adverse event occurred on the previous day, wherein said adverse event is selected from the group consisting of ssupplemental oxygen administration≧4 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, and the patient SpO2 while breathing room air is 90-92% and has decreased from a baseline of≧93% on the previous dosing day; and
ii) 100 mg/kg, if conditions are radiation treatment day 3-10, the dose was 75 mg/kg on the previous two dosing days or 100 mg/kg on the previous dosing day, and SpO2 while breathing room air is≧90% and no adverse event occurred on the previous dosing day, wherein said adverse event is selected from the group consisting of supplemental oxygen administration≧4 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, and the patient SpO₂ while breathing room air is 90-92% and has decreased from a baseline of≧93% on the previous dosing day; and
iii) 0 mg/kg, if conditions are conditions selected from the group consisting of:
a) SpO2 is<90%,
b) creatinine is>2.0 mg/dL;
c) the patient developed hypoxemia which required treatment on the previous treatment day;
d) RT day 2-10, the dose was 75 mg/kg on the previous day and an adverse event occurred, wherein said adverse event is selected from the group consisting of supplemental oxygen administration>3 hours after end-infusion of efaproxiral sodium before SpO2 while breathing room air returned to≧90% on the previous dosing day, the patient experienced nausea and/or vomiting (grade 2 or higher) or clinically significant hypotension associated with efaproxiral sodium within 12 hours after efaproxiral sodium administration on the previous dosing day, the patient developed hypoxemia which required treatment after discharge on the previous dosing day, and SpO2 while breathing room air is 90-92% but was≧93% on the previous dosing day; and
C) administering supplemental oxygen to the host,
wherein the radiation, supplemental oxygen, and efaproxiral sodium are administered in amounts effective to cause an arrest or regression of the central nervous system cancer in the host.

4. The method of claim 1, wherein the radiation is administered in at least about 3 Gray (Gy) fractions at least once every day for ten days to a treatment volume.

5. The method of claim 1, wherein the radiation is administered in fractions, wherein 10 fractions are administered to an initial treatment volume.

6. The method of claim 1, wherein a total of at least about 30 Gy of radiation is administered to the host.

7. The method of claim 1, wherein radiation is administered to a whole brain of the host.

8. The method of claim 1, wherein the efaproxiral sodium is administered via a route selected from the group consisting of intravenously including via a central venous access device, or via a peripheral route, via continuous infusion, and intraarterially.

9. The method of claim 1, wherein the efaproxiral sodium is administered at an initial dosing level of at least about 75 mg/Kg/day.

10. The method of claim 1, wherein the efaproxiral sodium is administered so as to achieve a RBC conentration of greater than about 483 μg/ml.

11. The method of claim 1, wherein the metastatic cancer is derived from a primary cancer selected from the group consisting of lung, breast, melanoma, renal, and colorectal.
